(19) United States
(12) Patent Application Publication
(10) Pub. No.: US 2024/0285171 A1
(43) Pub. Date: Aug. 29, 2024

(54) INSPECTION DEVICE FOR ORAL TISSUES OR DEPOSITS, AND INSPECTION AUXILIARY MEMBER

(71) Applicant: ShinSei Co., Ltd., Jyoyo-shi, Kyoto (JP)

(72) Inventors: Ryutaro ASHIDA, Jyoyo-shi (JP); Shota SOMEKAWA, Jyoyo-shi (JP); Michiya FUJIKI, Jyoyo-shi (JP); Sayaka MATSUZAWA, Jyoyo-shi (JP)

(73) Assignee: ShinSei Co., Ltd., Jyoyo-shi, Kyoto (JP)

(21) Appl. No.: 18/573,581

(22) PCT Filed: Mar. 22, 2022
(86) PCT No.: PCT/JP2022/013218

§ 371 Date: Dec. 22, 2023

(30) Foreign Application Priority Data
Jun. 24, 2021 (WO) PCT/JP2021/024004

(51) Int. Cl. A61B 5/00; A61B 8/12; A61C 19/06

(52) U.S. Cl. CPC A61B 5/0088; A61B 5/743; A61C 19/063; A61B 8/12

(57) ABSTRACT

This inspection device includes: a detection element for obtaining information on an oral tissue or deposits to be inspected; a case body that includes an information transmission line for transmitting information to the detection element; and an inspection auxiliary member that has a path or a guide through which the detection element or the information transmission line can move and is shaped to follow a tooth row.

### TECHNICAL FIELD

The present invention relates to an inspection device for oral tissues of a living body or deposits attached to the oral tissues, and an inspection auxiliary member.

### BACKGROUND ART

In recent years, as the coronavirus pandemics, aging of the society, and depopulation take place, limitations on movement have come to be imposed on those who wish to receive medical diagnosis and treatment, including dental care. In response to such social demands, online medical care, including dental diagnosis and treatment, has come to be practiced actively. In the current online practice related to dentistry, a pen-shaped intraoral camera is lent to a subject, and the subject captures an image of his/her own oral cavity.

However, such conventional pen-like intraoral cameras (see, for example, Patent Literatures 1 and 2) sometimes have difficulty in capturing image of a target tissue, due to lack of skills of the subject capturing an image, e.g., due to camera shakes. In addition, to capture an image of one tooth, for example, it is necessary to capture images from three sides, that is, from the lingual side, the buccal side, and in the occlusal side of the tooth, and sometimes the degree of burden on the subject has been a problem. For subjects who are children or elderly, it has been burdensome even to keep their mouth open to capture the image, to begin with.

In addition, in online dental practice, there have not been sufficient software prepared for efficiently sharing information of diagnosis results and making use of the information in the treatment.

### CITATION LIST

### Patent Literatures

Patent Literature 1: JP 2019-536553 W
Patent Literature 2: JP 2016-54590 A

### SUMMARY OF INVENTION

### Technical Problem

An object of the present invention is to provide a technology capable of assisting collection of information used in diagnosing and treating of oral tissues such as teeth. Another object of the present invention is to provide a technology for improving the efficiency of diagnoses and medical cares, by using the information of captured images of oral tissues.

### Solution to Problem

To solve the problems described above, the present invention may be an inspection device for oral tissues or deposits, the inspection device including:
a detection element for obtaining information on an oral tissue or a deposit that is an inspection target;
a case body internal of which is provided with the detection element or an information transmission line for transmitting information to the detection element; and
an inspection auxiliary member that includes a path or a guide along which the detection element or the information transmission line is movable, and that has a shape following a dentition.

With this, by having the subject hold and fix the inspection auxiliary member between his/her dentitions, and move the detection element along the path or the guide of the inspection auxiliary member, it is possible to obtain information on an oral tissue easily. Examples of the oral tissues or the deposits include healthy teeth, caries, demineralization, caries, microcracks, microvoids, calculus, dental plaque, and gingiva. Examples of the detection element include a light receiving element that obtains intensity of light at a specific wavelength, an imaging element that captures an image, and an acoustic wave detection element that detects an acoustic wave or an ultrasonic wave. Note that moving the detection element herein includes moving the detection element or the case body (described later) having the detection element mounted thereon along the path or the guide. An example of moving the information transmission line includes moving the tip of the optical fiber, with the detection element disposed outside, along the path or the guide, for example.

In the above description, the inspection auxiliary member may have a transmitting portion or a gap that transmits light or an acoustic wave from the oral tissue or the deposit. With this, because light or an acoustic wave can be detected through the inspection auxiliary member, the degree of freedom in designing the inspection auxiliary member and the case body can be improved. In the present invention, light basically means light within a range of near-infrared light having a wavelength of 1200 nm and ultraviolet light having a wavelength of 250 nm. However, this description is not intended to exclude any light within a wider wavelength range, e.g., X-rays or far-infrared light.

The configuration described above may further include a light source or an oscillator element disposed inside or outside the case body. With this, it is possible to detect reflected light, scattered light, fluorescence, luminescence, or the like, by irradiating the oral tissues with light, as well as to control the luminance of an image to be captured. In such a configuration, the detection element may be an element that detects reflected light, scattered light, fluorescence, luminescence, or the like from the oral tissue or the deposit. In addition, the number of the detection elements may be one or more. Furthermore, the detection element may constitute only of the element, or may also include an optical element such as a lens. Furthermore, in the above configuration, it is possible to disinfect the oral tissues, by irradiating the oral tissue with ultraviolet light or the like emitted from the light source.

The above configuration may also include a light source or an oscillator element disposed inside or outside the case body, and
an oral cavity may be irradiated with light from the light source or an acoustic wave from the oscillation element, via the path or the guide.

Furthermore, in the configuration described above, the inspection auxiliary member or the case body may be made from at least any one of a predetermined resin, ceramic, and metal that are safe for the oral tissues. With this, even when the subject hold and fixes the inspection auxiliary member between his dentitions, it is possible to keep the sanitary risk of the subject low.

Furthermore, in the configuration described above, the case body may further include a directing portion that reflects or refracts light or an acoustic wave from the oral tissue or the deposit that is the inspection target and directs the light or the acoustic wave to the detection element. With this, the degree of freedom in the paths of light, acoustic waves, and the like emitted to the oral tissues can be improved.

Furthermore, in the configuration described above, the inspection device may further include an image processing unit that generates an image or a spectrum of the oral tissue or the deposit that is the inspection target, based on an output signal from the detection element;
a determination unit that determines or aids determination of presence of a disease of an oral tissue that is the inspection target including a tooth, based on the image or the spectrum generated by the image processing unit; and
a display unit that displays the image or the spectrum generated by the image processing unit and/or a determination result of the determination unit.

Furthermore, in the configuration described above, the detection element may be an imaging sensor that captures an image of the oral tissue that is the inspection target. With this, a high-quality image of the oral tissue or the deposit that is an inspection target can be obtained more easily.

Furthermore, in the configuration described above, the case body may be movable inside the inspection auxiliary member along the path or the guide in the inspection auxiliary member, and
the detection elements may be provided on a front side and lateral sides, respectively, with respect to a movement of the case body. With this, information of the three sides of the oral tissue or the deposit can be obtained simultaneously.

Furthermore, in the configuration described above, the case body may include a directing portion that is provided on a front side of the case body, being on the front side with respect to the movement, and that reflects or refracts light or an acoustic wave from the oral tissue or the deposit that is the inspection target and guides the light or the acoustic wave to the detection element, and
the inspection auxiliary member may include lateral directing portions that are provided to parts of the path or the guide in a manner facing the lateral sides of the case body, respectively, being on the lateral sides with respect to the movement, and that reflect or refract light or an acoustic wave from the oral tissue or the deposit that is the inspection target and guides the light or the acoustic wave to the detection element. With this, by the detection elements, and the directing portions and the lateral directing portions provided on the front side and the lateral sides with respect to the movement of the case body, it is possible to obtain information such as an image of the oral tissue or the deposit, from the upper front side and the lower front side of the case body, and from the upper lateral side and lower lateral side on the lateral sides of the case body, at once.

Furthermore, in the configuration described above, the detection element provided on the front side of the case body is installed facing a direction in which the case body is advanced, and the detection elements provided on the respective lateral sides of the case body are installed inclined with respect to an up-down direction of the case body, and
the directing portion, provided on the front side of the case body with respect to the movement, may be installed inclined with respect to the up-down direction of the case body, and the lateral directing portions, provided to parts of the path or the guide of the inspection auxiliary member in a manner facing the lateral sides of the case body, respectively, being on the lateral sides with respect to the movement, may be installed in an orientation in which normal lines of the lateral directing portions extend horizontally.

With this, it is possible to obtain images captured from the up-down direction of the oral tissue, using the detection element provided in front of the case body, and to obtain images of the oral tissue captured diagonally, from the upper and lower lateral sides, using the detection elements and the lateral directing portions provided on the lateral sides of the case body. Furthermore, it is possible to obtain these images simultaneously. Note that the inclination angle of the detection element and the directing portion installed inclined with respect to the up-down direction may be 45 degrees ±5 degrees. With this, vertical light can be converted into horizontal light, for example. However, the inclination angle is not limited to the angle mentioned above, and may be determined as appropriate, depending on the positional relationship between the detection element and the oral tissues or the deposit, and the shape and dimension of the inspection auxiliary member or the case body. In addition, in the configuration described above, the inclination with respect to the up-down direction does not exclude inclination with respect to the left-right direction. The inclinations with respect to the up-down direction and the left-right direction are included.

Furthermore, the configuration described above may further include:
an image processing unit that generates an image of the oral tissue or the deposit that is the inspection target, based on an output signal from the detection element; and
a display unit that displays the image generated by the image processing unit, wherein
the oral tissue may be a dentition,
the image processing unit may generate a map image that is a combination of a linear image obtained by joining images of the dentition captured from a buccal side and/or a lingual side of the dentition to a linear image, and a planar image obtained by joining images of the dentition captured from an occlusal side, the images being captured using the detection element and the inspection auxiliary member, to a planer image, and
the display unit may display the map image. With this, the images of the oral tissues can be displayed in a manner that is more easily understandable, so that the conditions of the oral tissues can be compared with other examples under the same condition. Therefore, the diagnosis of the oral tissues can be made more efficiently.

In the above description, the path or the guide of the inspection auxiliary member may further include an ejector portion that ejects to supply a predetermined liquid or gel substance onto the oral tissue. With this, it is possible to eject to supply water or a medicinal solution from the inspection auxiliary member onto the oral tissue that is the inspection target, and to perform treatment on the spot.

In the configuration described above, the case body may include a propulsion mechanism for moving the case body along the path or the guide. With this, it is possible to make it easier to move and to control the position of the case body, the detection element, or the information transmission line, and it becomes possible for the inspector or the subject to concentrate on obtaining information such as an image.

Furthermore, in the configuration described above, the case body may move along the path or the guide of the inspection auxiliary member, and
a position information detection unit capable of obtaining position information of the case body inside the path or the guide may be further provided. With this, it is possible to easily obtain information indicating from which oral tissue the detection element has obtained information, as an aid for subsequent information analysis and mapping.

Furthermore, in the configuration described above, the detection element may include a fisheye lens or a super wide-angle lens capable of capturing an image across a wider range. With this, it is possible to increase the range in which one detection element can capture an image, so that it becomes possible to reduce the number of detection elements.

Furthermore, in the configuration described above, the detection elements may be provided at a plurality of positions on the case body. With this, the detection element to make a detection can be switched, depending on the position and the side of the oral tissues or the deposit that is the inspection target, and the range of the positions and sides of the inspection target can be broadened. In addition, it is possible to obtain information on a plurality of ranges of the inspection target at once, so that inspection efficiency can be improved. Note that the orientation of the detection element in this example may be inclined in any of upwards, downwards, leftwards, and rightwards, or may be directed horizontally or vertically.

In the configuration described above, the inspection auxiliary member may include a directing portion that reflects or refracts light or an acoustic wave from the oral tissue or the deposit that is the inspection target and guides the light or the acoustic wave to at least one of the detection elements provided at the plurality of respective positions of the case body. With this, by selecting a combination of the detection elements and the directing portions appropriately, it is possible to cover a broader range of positions and directions of the inspection target.

In addition, the present invention may be an inspection auxiliary member including a detection element that obtains information of an oral tissue or a deposit that is an inspection target, or a path or a guide through which an information transmission line is movable, and having a shape following a dentition.

The inspection auxiliary member may further include a transmitting portion or a gap that transmits light or an acoustic wave from the oral tissue or the deposit.

In the inspection auxiliary member, the detection element may move along the path or the guide, and the inspection auxiliary member may further include a mirror surface that reflects light to be detected by the detection element, on a part of the shape following the dentition. With this, by adjusting the position and angle of the mirror surface, it is possible to adjust the information to be obtained by the detection element, and the positions for which the images are to be captured by the imaging element, variously.

In the present invention, it is possible to use the means for solving the problems described above in combinations as many as possible.

### Advantageous Effects of Invention

According to the present invention, it is possible to collect information used in diagnosis and treatment of oral tissues such as teeth, more easily. In addition, it is possible to improve the efficiency in making diagnoses and providing medical care, by using the collected information on the oral tissues.

### BRIEF DESCRIPTION OF DRAWINGS

FIG. 1A, FIG. 1B, FIG. 1C and FIG. 1D are schematic diagrams of a guide member according to a first embodiment of the present invention.

FIG. 2A, FIG. 2B and FIG. 2C are schematic diagrams of an imaging unit according to the first embodiment of the present invention.

FIG. 3A and FIG. 3B are diagrams for explaining a movement of the imaging unit in the guide member according to the first embodiment of the present invention.

FIG. 4 is a block diagram illustrating the overall configuration of an inspection device according to the first embodiment of the present invention.

FIG. 5A, FIG. 5B, FIG. 5C and FIG. 5D are schematic diagrams illustrating a guide member according to a second embodiment of the present invention.

FIG. 6A, FIG. 6B and FIG. 6C are schematic diagrams of an imaging unit according to a third embodiment of the present invention.

FIG. 7A, FIG. 7B, FIG. 7C and FIG. 7D are schematic diagrams illustrating a guide member according to a third embodiment of the present invention.

FIG. 8 is a schematic diagram illustrating an example of a map in the third embodiment of the present invention.

FIG. 9A, FIG. 9B and FIG. 9C are schematic diagrams of an imaging unit in a fourth embodiment of the present invention.

FIG. 10A, FIG. 10B, FIG. 10C and FIG. 10D are schematic diagrams illustrating a guide member according to the fourth embodiment of the present invention.

### DESCRIPTION OF EMBODIMENTS

Embodiments of the present invention will now be explained with reference to drawings. Note that the embodiments described below provides examples for carrying out the present invention, and the present invention is not limited to the specific configurations described below.

### First Embodiment

A first embodiment of the present invention will now be explained. In oral tissues inspections, it is preferable to capture actual images of the oral tissues that are the inspection target. Explained below is a device used in capturing an actual image of the oral tissue that is the inspection target, and an inspection system that determines or assists determination on the presence of diseases such as caries, demineralization, proximal caries, microcracks, microvoids, calculus, or dental plaque, from information including the obtained actual image.

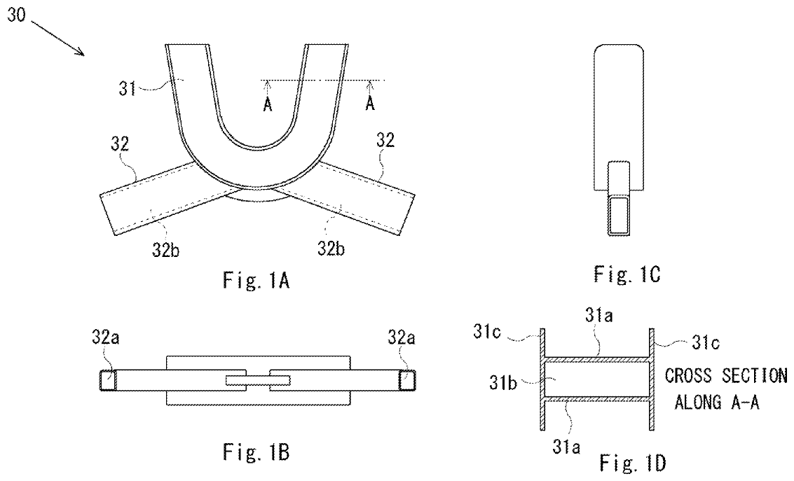

FIG. 1A, FIG. 1B, FIG. 1C and FIG. 1D are schematic diagrams of a guide member 30, which is an example of an inspection auxiliary member, used in obtaining an image of a dentition, which is an example of the oral tissue that is the inspection target. FIG. 1A is a plan view, FIG. 1B is a front view, and FIG. 1C is a side view of the guide member 30. FIG. 1D is a cross-sectional view taken along the line A-A in FIG. 1A. The guide member 30 is a device held inside the mouth of the subject, between his/her dentitions, when an actual image of the oral tissue is to be obtained. An imaging sensor that is a light receiving element is moved inside the guide member 30, and captures an image. The guide member 30 includes a mouthpiece portion 31 to be held inside the mouth of the subject, and inside of which the imaging sensor moves, and an inserting and removing portion 32 via which the imaging sensor is inserted into or removed from the mouthpiece portion 31. The guide member 30 is made from, for example, a thermoplastic resin material, such as polycarbonate, polyethylene, ABS, siloxane, polystyrene, or acrylic, a thermosetting resin material, such as epoxy, or a photocurable resin material.

The mouthpiece portion 31 has a curved shape similar to that of a dentition in a plan view. As illustrated in FIG. 1D, a tunnel-like path 31b having a rectangular cross section is formed inside the mouthpiece portion 31. The path 31b corresponds to a travel path, a path, or a guide. The top surface and the bottom surface of the path 31b in the orientation during the use include fixing portions 31a enabling the guide member 30 to be fixed, by being held inside of the mouth of the subject, between his/her maxillary and mandibular dentitions. In addition, provided on ends of the path 31b in the horizontal direction are walls 31c by which the dentitions are covered from the buccal side (labial side) and the lingual side (pharynx side), respectively, when the subject holds the fixing portion 31a inside his/her mouth.

The inserting and removing portions 32 are tunnel-shaped members each having an entry path 32b the shape of which is substantially the same as that of the path 31b. One end of the inserting and removing portion 32 is connected to the mouthpiece portion in such a manner that the entry path 32b is connected to the path 31b, and the other end has an opening 32a. The entry path 32b of the inserting and removing portion 32 on the left side in FIG. 1A is connected to the path 31b of the right half of the mouthpiece portion 31, at an angle allowing these paths to communicate each other smoothly. Similarly, the entry path 32b of the inserting and removing portion 32 on the right side is connected to the path 31b of the left half of the mouthpiece portion 31, at an angle allowing these paths to communicate each other smoothly.

The path 31b and the entry paths 32b are configured in such a manner that the imaging unit 40, which is to be described later, is allowed to pass therethrough. In other words, the imaging unit 40 is guided into the guide member 30 via the inserting and removing portion 32, and captures images of the maxillary and/or mandibular dentition, in the orientation during the use, while being moved along the path 31b. The fixing portion 31a may therefore be made from a light-transmitting resin. Portions made from a light-transmitting resin correspond to transmitting portions. The walls 31c may be made from a colored resin, to remove the stray light. Furthermore, the fixing portion 31a may have a gap for allowing the light passed through the gap to reach the imaging unit 40. In such a configuration, the gap corresponds to the transmitting portion.

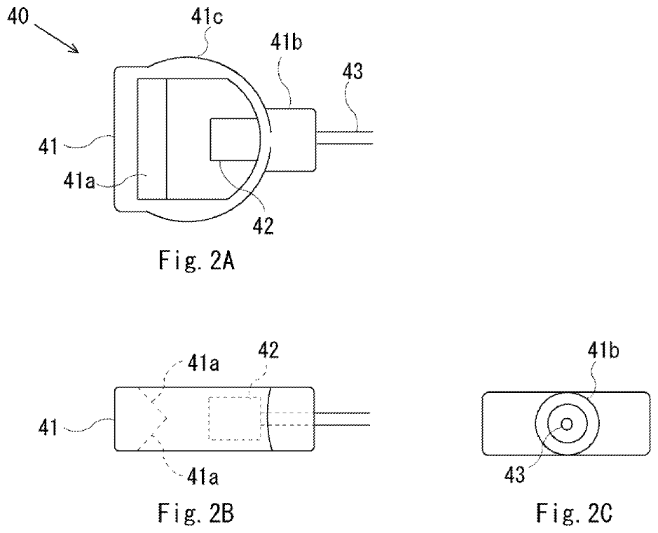

FIG. 2A, FIG. 2B and FIG. 2C are schematic diagrams of the imaging unit 40 according to the present embodiment. FIG. 2A is a plan view, FIG. 2B is a side view, and FIG. 2C is a rear view. The imaging unit 40 includes a unit body 41 as an example of a case body or a case main body, an imaging sensor 42 as an example of a detection element or a light receiving element, and an input/output cable 43 as an example of an information transmission line. A front portion of the unit body 41 has a substantially rectangular shape in a plan view, and a rear portion has a curved portion 41c with a diameter slightly smaller than the width of the path 31b of the guide member 30 and the entry path 32b of the inserting and removing portion 32. The imaging unit 40 also includes an opening tubular portion 41b where the imaging sensor 42 is fixed, and through which the input/output cable 43 is passed, and mirrors 41a reflecting the light in the up-down direction by 90 degrees, so that the light becomes incident on the imaging sensor 42. Similarly to the guide member 30, the unit body 41 is made from a thermoplastic resin material such as polycarbonate, polyethylene, ABS, siloxane, polystyrene, or acrylic, a thermosetting resin material such as epoxy, or a photocurable resin material. For each of the mirrors 41a, an optical mirror may be pasted or coating of a reflective film may be provided on a resin surface, using a method such as vapor deposition or plating. Note that the unit body 41 and the guide member 30 may be made from other types of resin, ceramic, or metal as long as such a material is safe for the oral tissues. Note that the imaging sensor 42 may include a fisheye lens or a super wide-angle lens to broaden the imaging range.

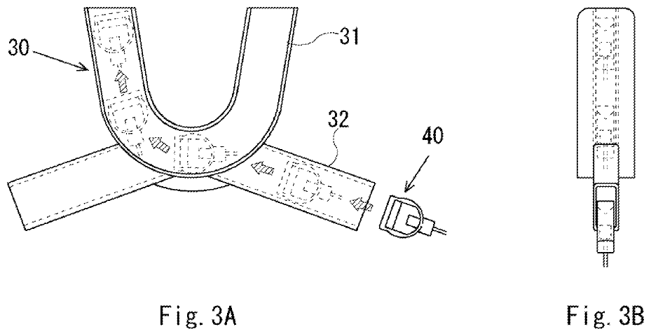

When the subject is to obtain captured images of the actual dentitions, the imaging unit 40 is inserted via the opening 32a of the inserting and removing portion 32 of the guide member 30, and goes into the entry path 32b, as illustrated in FIG. 3A and FIG. 3B. The inspector then pushes the imaging unit 40 deeper into the inserting and removing portion 32, using the input/output cable 43. The imaging unit 40 is then moved into the path 31b of the fixing portion 31a.

The inspector then further advances the imaging unit 40 by pushing the input/output cable 43, so as to move the imaging unit 40 in the path 31b, in a manner following the dentition. At this time, the light from the maxillary and mandibular dentitions is reflected by the mirrors 41a and becomes incident on the imaging sensor 42, which, in turn, captures images of the maxillary and mandibular dentitions. The imaging sensor 42 performs photoelectric conversion of the electric signals, and transmits the resultant signals to an image processing unit 35 (described later) via the input/output cable 43, and image data is generated thereby.

The unit body 41 of the imaging unit 40 has curved portions 41c with a diameter slightly smaller than the width of the path 31b of the guide member 30 and the entry path 32b of the inserting and removing portion 32. In this manner, the imaging unit 40 can advance into the entry path 32b and the path 31b stably, and move across the boundary between the entry path 32b of the inserting and removing portion 32 and the path 31b of the fixing portion 31a, and along the curved surface of the path 31b, smoothly.

As illustrated in FIG. 3A, when the imaging unit 40 is inserted into the inserting and removing portion 32 on the right side, the imaging unit 40 goes into the path 31b of the left half of the mouthpiece portion 31. Similarly, when the imaging unit 40 is inserted into the inserting and removing portion 32 on the left side, the imaging unit 40 goes into the path 31b of the right half of the mouthpiece portion 31. With this, when the imaging unit 40 completes capturing images of the dentition on the fixing portion 31a on the left half of the guide member 30 illustrated in FIG. 3A, the imaging unit 40 is inserted into the inserting and removing portion 32 again but on the left side, via the opening 32a on the left side in the drawing, and captures images of the dentition on the fixing portion 31a on the right half of the guide member 30.

Note that the imaging unit 40 or the guide member 30 may be enabled to obtain position information of the imaging unit 40 inside the guide member 30. More specifically, the guide member 30 may be provided with some index for allowing the position information to be included in a part of an image of the dentition captured by the imaging unit 40. The imaging unit 40 may also be configured to optically or electrically read the position information provided on the guide member 30. Because various conventional techniques are available as to how the position information is provided and read by the imaging unit 40, the description thereof will be omitted herein. Alternatively, in a configuration in which the imaging unit 40 has wheels, as will be described later, the position information may be obtained by measuring the rotational angle of the wheels. Furthermore, the position of the imaging unit 70 may be obtained from satellite information.

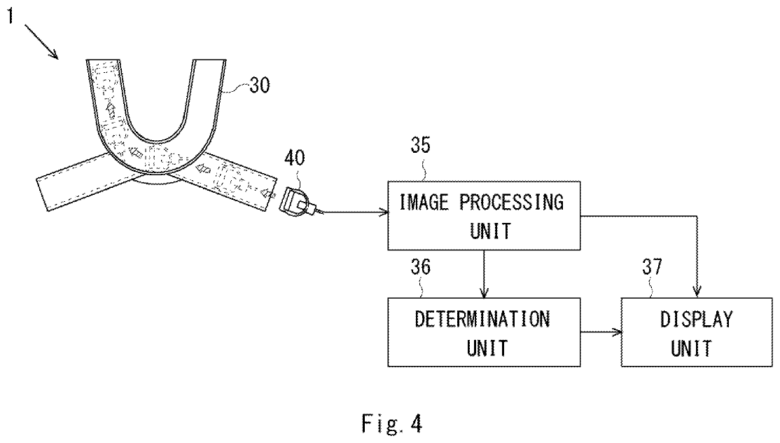

FIG. 4 is a schematic of the overall inspection device 1 according to the present embodiment. An image (including a 3D image and a panoramic image) captured using the guide member 30 and the imaging unit 40 is transmitted to the image processing unit 35 via the input/output cable 43. The image processing unit 35 generates images of the maxillary and mandibular dentitions, by joining the images captured at the respective positions of the guide member 30. At that time, the image processing unit 35 may also generate a map resultant of developing the actual images onto a plane, according to a specific rule, instead of the actual images, or in addition to the actual images. The images and the map of the dentitions are transmitted to and displayed on a display unit 37, and are also transmitted to a determination unit 36.

On the basis of the image or the map image of the dentitions generated by the image processing unit 35, the determination unit 36 determines or aids determination of the presence of any disease, such as caries, demineralization, adjacent surface caries, microcrack, microvoids, calculus, or dental plaque, on each tooth. In making the determination or aiding the determination, the determination unit 36 may use a learner trained to determine the presence of a disease such as caries, demineralization, caries between adjacent teeth, microcracks, microvoids, calculus, or dental plaque, from images or maps of teeth, in advance. In addition, the determination unit 36 may make a rule-based determination according to a predetermined rule. In addition, the determination unit 36 make a clear determination result as to whether the disease is present, or only aid determination, e.g., by presenting an image and the probability of the presence of each type of diseases. The result of the determination made by or aided by the determination unit 36 is displayed on the display unit 37. The image processing unit 35, the determination unit 36, and the display unit 37 in the present embodiment may be implemented by a processor of a general PC, for example. Note that, explained in the present embodiment is an example in which the inspection device 1 makes or aids determination as to whether there is any disease such as caries, demineralization, adjacent surface caries, microcracks, microvoids, calculus, or dental plaque, on each tooth, on the basis of an image (including an 3D image or a panoramic image) captured using the guide member 30 and the imaging unit 40. However, in addition to the use of an image, the determination unit 36 may make or aid determination as to whether any disease is present, on the basis of the patterns of a spectrum of detection light obtained from the teeth, instead of or in combination with the images. In addition, the inspection target is not limited to the teeth, and may be another type of oral tissues such as gingiva.

### Second Embodiment

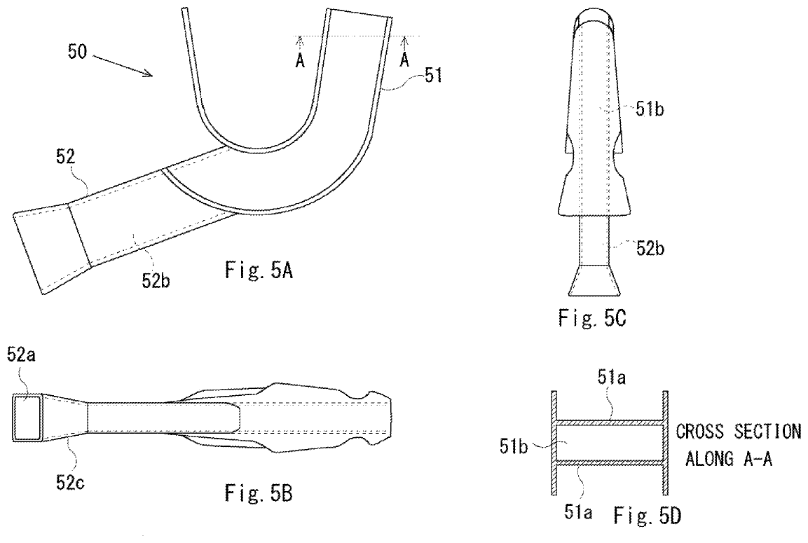

FIG. 5A, FIG. 5B, FIG. 5C and FIG. 5D illustrate a second embodiment of the guide member described in the present embodiment. FIG. 5A, FIG. 5B, FIG. 5C and FIG. 5D are schematic diagrams of a guide member 50. FIG. 5A is a plan view, FIG. 5B is a front view, and FIG. 5C is a side view. The guide member 50 includes a mouthpiece portion 51 and an inserting and removing portion 52. The mouthpiece portion 51 has a curved shape similar to that of a dentition in a plan view. The mouthpiece portion 51 is different from the mouthpiece portion 31 in that fixing portions 51a and a path 51b of the mouthpiece portion 51 are provided only to a half of the dentition. The subject then fixes the guide member 50 by holding the fixing portion 51a of the path 51b between the dentitions, on one side that is the inspection target (the left half in FIG. 4). After completing capturing an image of the dentitions on the one side of the inspection target, the guide member 50 is reversed, and an image of the dentitions on the opposite side is then captured.

With this structure, upon completing capturing images of the dentition on one side that is subjected to a first inspection, it is possible to reverse the guide member 50, with the imaging unit 40 held inside the guide member 50, and then capture an image of the dentition on the opposite side. In other words, it is possible to omit the steps for taking out the imaging unit 40 from the guide member 50, and inserting the imaging unit 40 again via the inserting and removing portion on the opposite side. In the present embodiment, an enlarged portion 52c where the entry path 52b has a larger size in the up-down direction and the left-right direction is provided near the opening 52a of the inserting and removing portion 52. With this, the imaging unit 40 can be inserted more smoothly into the inserting and removing portion 52.

Explained in the embodiment is an example in which the inspector moves the imaging unit 40 inside the guide member 30, 50 using the input/output cable 43 of the imaging unit 40, but the means for controlling the position of the imaging unit 40 in the guide member 30, 50 is not limited thereto. For example, a moving mechanism may be provided inside the guide member 30, 50 and/or the inserting and removing portion 32, 52. One example includes a wire-type moving mechanism for pushing and pulling a wire to which the imaging unit 40 is fixed, between the deepest part of the path 31b, 51b and the opening 32a, 52a. Furthermore, an electromagnetically driven actuator such as a motor may be mounted on the imaging unit 40 itself.

Furthermore, explained in the embodiment above is an example of the imaging unit 40 including the mirrors 41a having two mirrors that reflect light becoming incident thereon from above and below, respectively. However, but needlessly to say, a mirror having one mirror that reflects light from one of the above and the below may be provided, instead of the mirrors 41a. In addition, instead of the mirrors 41a, the light from the above and the below may be guided to the imaging sensor 42 using an optical fiber. Furthermore, the imaging sensor 42 may be mounted on the imaging unit 40 by changing the inclination of the imaging sensor 42 by 90 degrees so that the optical axis direction of the imaging sensor extends in one of upwards and downwards.

Furthermore, in the present embodiment, the imaging unit 40 has been explained assuming that the image of the maxillary dentition is captured from below, and the image of the mandibular dentition is captured from above. However, the imaging unit 40 may be enabled to capture images of maxillary and mandibular dentitions or other oral tissues, from the buccal side or the lingual side of the dentitions, by disposing mirrors on the mouthpiece portion 31, 51, appropriately. Furthermore, the imaging unit 40 may include a predetermined optical system, in addition to the mirrors 41a, to enable the area from which image can be captured to be changed, as appropriate. For example, a fisheye lens may be provided to broaden the angle of view.

For the use in the actual inspection, at least the guide member 30, 50 may be made disposable. In this manner, it is possible to always ensure the hygiene of inspections of oral tissues and to keep the risk of infections such as contagious diseases extremely low.

Furthermore, explained in the embodiment is an example in which an image is generated by irradiating oral tissues such as teeth with the light emitted from the imaging unit 40, and causing the imaging sensor 42 of the imaging unit 40 to detect the detection light from the oral tissue. However, it is also possible to dispose the imaging sensor 42 outside the imaging unit 40. For example, the imaging sensor 42 may be disposed outside of the guide member 30, 50, and the light from the imaging unit 40 may be transmitted to the imaging sensor 42 via an optical fiber that is the information transmission line. Furthermore, explained in the embodiment is an example in which an image is formed optically by causing the imaging sensor 42 to detect the detection light from oral tissues such as teeth. However, it is also possible, depending on situations, to generate an image by emitting an acoustic wave or a pulse wave from the imaging unit 40 to the oral tissues, and causing an acoustic sensor to detect the acoustic wave or the shock wave from the oral tissues such as teeth, in the same manner. In such a configuration as well, the inspection device is operated in the same manner as that using light, and therefore, detailed description thereof will be omitted herein.

### Third Embodiment

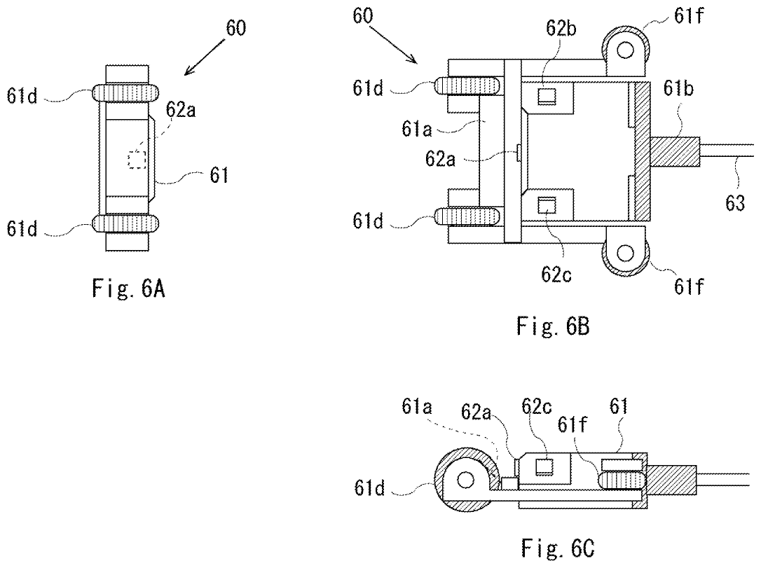

A third embodiment of the present invention will now be explained. FIG. 6A, FIG. 6B and FIG. 6C are schematic diagrams of the imaging unit 60 according to the present embodiment. FIG. 6A is a front view, FIG. 6B is a plan view, and FIG. 6C is a side view. The imaging unit 60 includes a unit body 61 as an example of the case body or the case main body, and imaging sensors 62a to 62c as an example of the detection elements or the light receiving elements. The imaging sensor 62a is provided on the front surface of the unit body 61, and the optical axis direction is matched with the direction in which the unit body 61 is advanced. The imaging sensors 62b, 62c are mounted on respective sides in the left-right direction, with respect to the direction in which the unit body 61 is advanced, and are provided in a manner inclined by 45 degrees with respect to the up-down direction. With the image information from the three imaging sensors 62a to 62c, images at three sides of the buccal side, the lingual side, and the occlusal side of the tooth can be obtained at once.

Wheels 61d having their rotation axis in the left-right direction are rotatably mounted on the front surface of the unit body 61. Wheels 61f having their rotation axis in the up-down direction are rotatably mounted on the left and right side surfaces of the unit body 61, respectively. Therefore, when the imaging unit 60 moves along the path of the guide member, the resistance in the movement can be reduced, so that the imaging unit 60 can be moved more smoothly. Furthermore, even when the imaging unit 60 comes into contact with the side surface of the path as the imaging unit 60 moves, the imaging unit 60 can be moved smoothly.

The imaging unit 60 also includes a mirror 61a that reflects light becoming incident from above, by 90 degrees so as to enable the light to become incident on the imaging sensor 62a. The imaging unit 60 also has an opening tubular portion 61b through which an input/output cable 63, as an example of the information transmission line, is passed, and the input/output cable 63 is connected to the imaging sensors 62a to 62c inside or on a surface of the unit body 61. Note that some or all of the imaging sensors 62a to 62c illustrated in FIG. 6A, FIG. 6B and FIG. 6C may include a fisheye lens or a super wide-angle lens to broaden the imaging range.

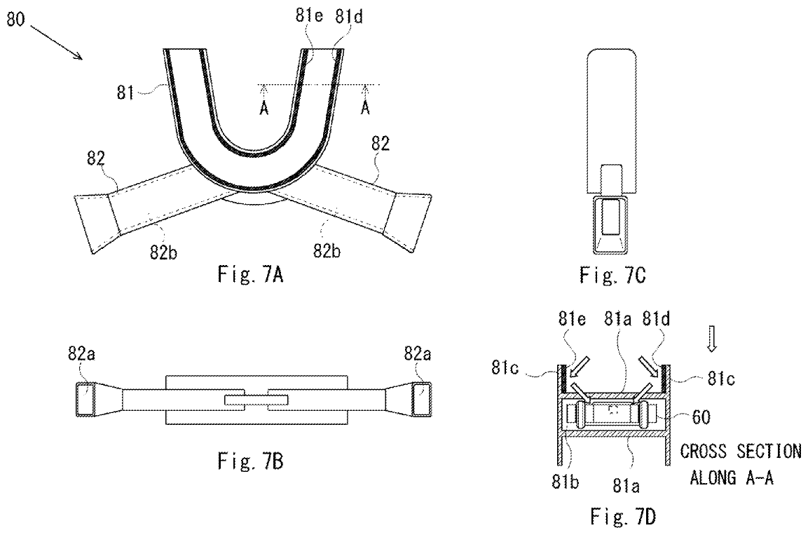

FIG. 7A, FIG. 7B, FIG. 7C and FIG. 7D are schematic diagrams of a guide member 80 that is the inspection auxiliary member according to the present embodiment. FIG. 7A is a plan view of the guide member 80, FIG. 7B is a front view, and FIG. 7C is a side view. FIG. 7D is a cross-sectional view taken along the line A-A in FIG. 7A. In the same manner as the guide member 30 described in the first embodiment, the guide member 80 is a device held inside the mouth of the subject, between his/her dentitions when an image of the actual oral tissue is to be obtained. The imaging unit 60 is moved inside the guide member 80, and captures an image. The guide member 80 includes a mouthpiece portion 81 and an inserting and removing portion 82.

As illustrated in FIG. 7D, a tunnel-like path 81b having a rectangular cross section is formed inside the mouthpiece portion 81. The top surface and the bottom surface of the path 81b in the orientation during the use include fixing portions 81a held between the maxillary and mandibular dentitions of the subject in his/her mouth. On horizontal ends of the path 81b and the fixing portion 81a, walls 81c are provided, so as to face the left and right side surfaces of the dentitions when the fixing portion 81a is held between the dentitions. Particularly, on the right and left ends of the fixing portion 81a, lateral mirrors 81d, 81e for reflecting the light from the dentitions and guiding the light to the imaging sensors 62b, 62c are provided.

The lateral mirrors 81d, 81e allow the imaging sensor 62b and the imaging sensor 62c to capture images of the buccal side and the lingual side of the dentition, which is the inspection target, respectively. Note that, as described earlier, the imaging sensors 62b, 62c provided in the left-right lateral direction of the imaging unit 60 are mounted in a manner inclined upwards by 45 degrees in FIG. 7D. The lateral mirrors 81d, 81e are pasted vertically on the walls 81c, respectively, as illustrated in FIG. 7D. Therefore, as illustrated in FIG. 7D, it is possible to capture the images of the dentition further above the fixing portion 81a, favorably.

In addition, as illustrated in FIG. 7A, the lateral mirrors 81d, 81e can be easily manufactured by bending and pasting members made from flexible material with a mirror-finished surface, on the inner surface of the outer circumferential side wall 81c and the lingual side of the inner circumferential side wall 81c of the fixing portion 81a, respectively.

Note that the inserting and removing portion 82 in the present embodiment has the same function as the inserting and removing portion 32 according to the first embodiment, but the inserting and removing portion 82 may also be made from a colored resin that blocks the external light. Note that the inspection device 1 illustrated in FIG. 4 may be configured by using the imaging unit 60 and the guide member 80 in the present embodiment, in addition to the image processing unit 35, the determination unit 36, and the display unit 37. When the determination is made by the inspector him/herself, the determination unit 36 is not necessarily required.

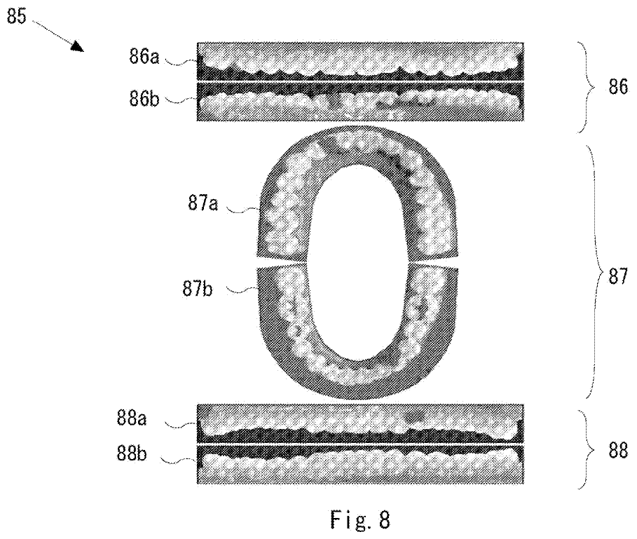

FIG. 8 illustrates an example of a map 85 obtained using the imaging unit 60 and the guide member 80, applied with the image processing of the image processing unit 35 and displayed on the display unit 37 that are illustrated in FIG. 4. As illustrated in FIG. 8, the map has three image areas: maxillary lingual/buccal images 86, occlusal images 87, and mandibular lingual/buccal images 88. The maxillary lingual/buccal images 86 include a maxillary buccal image 86a and a maxillary lingual image 86b. The occlusal images 87 include a maxillary occlusal image 87a and a mandibular occlusal image 87b. The mandibular lingual/buccal images 88 include a mandibular lingual image 88a and a mandibular buccal image 88b.

Each of the maxillary lingual/buccal images 86 and the mandibular lingual/buccal images 88 is an image resultant of causing the image processing unit 35 to join the images captured by the imaging sensor 62b, 62c disposed on the corresponding side of the imaging unit 60 into a linear image. With these, it is possible to make a diagnosis for the buccal side and the lingual side of the maxillary dentition and the mandibular dentition at once. Note that the example illustrated in FIG. 7A, FIG. 7B, FIG. 7C and FIG. 7D are arrangements for capturing the maxillary lingual/buccal images 86, but when the mandibular lingual/buccal images 88 are to be captured, the imaging unit 60 and the guide member 80 may be reversed upside down before use.

The occlusal images 87 are images resultant of causing the image processing unit 35 to join the images captured by the imaging sensor 62a disposed in front of the imaging unit 60 into a planer image. With these, it is possible to make a diagnosis for the occlusal surfaces of the dentitions at once. Note that there may be a function, when some marking is given to a specific tooth in any one of the maxillary lingual/buccal images 86, the occlusal images 87, and the mandibular lingual/buccal images 88 in the map 85, for example, automatically giving the marking to the same tooth in the image areas of the other images of the maxillary lingual/buccal images 86, the occlusal images 87, and the mandibular lingual/buccal images 88.

### Fourth Embodiment

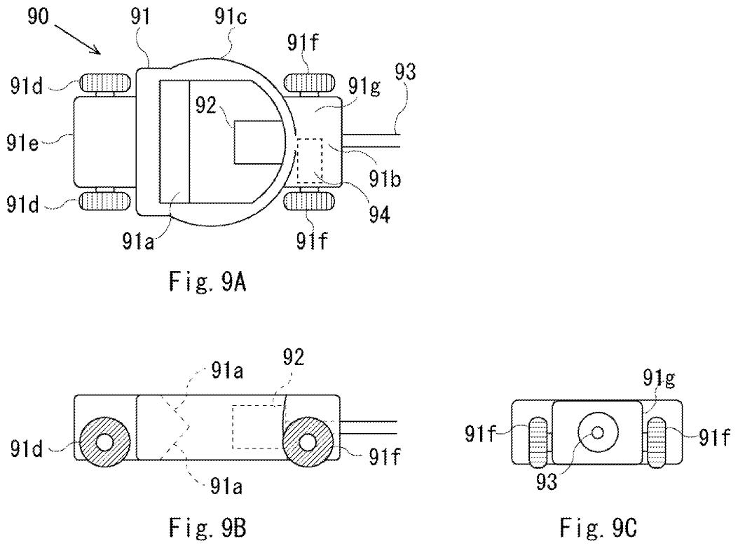

A fourth embodiment of the present invention will now be explained with reference to FIG. 9A, FIG. 9B and FIG. 9C. Explained in the fourth embodiment is an example in which the imaging unit is not moved manually by the inspector (e.g., by pushing and pulling the input/output cable), but moved by a drive mechanism included in the imaging unit. FIG. 9A, FIG. 9B and FIG. 9C are schematic diagrams of an imaging unit 90 according to the present embodiment. FIG. 9A is a plan view, FIG. 9B is a side view, and FIG. 9C is a rear view. The imaging unit 90 includes a unit body 91 as an example of the case body or the case main body, an imaging sensor 92, and an input/output cable 93 as an example of the information transmission line. The unit body 91 also includes an opening tubular portion 91b where the imaging sensor 92 is fixed, and through which the input/output cable 93 is passed, and mirrors 91a on which the light becoming incident in the up-down direction is reflected by 90 degrees, so that the light becomes incident on the imaging sensor 92. The unit body 91 also includes, in front of the mirrors 91a, wheels 91d facilitating the movement of the imaging unit 90, and a wheel holder 91e that rotatably holds the wheels 91d.

Driving wheels 91f and a driving mechanism holder 91g that holds a drive actuator 94 for driving the driving wheels 91f are provided behind the imaging sensor 92 of the imaging unit 90. In the imaging unit 90 according to the present embodiment, the input/output cable 93 has a function for supplying a driving signal to the drive actuator 94, in addition to a function for exchanging information and a driving signal with the imaging sensor 92. By the propulsive force generated by the driving of the driving wheels 91f, the imaging unit 90 can be moved along the path in the guide member. As a result, the inspector or the subject can concentrate on obtaining information including an image, so that the inspector or the subject can obtain the information on the oral tissues more easily. Examples of the drive actuator 94 include a small DC motor and an ultrasonic motor.

### Fifth Embodiment

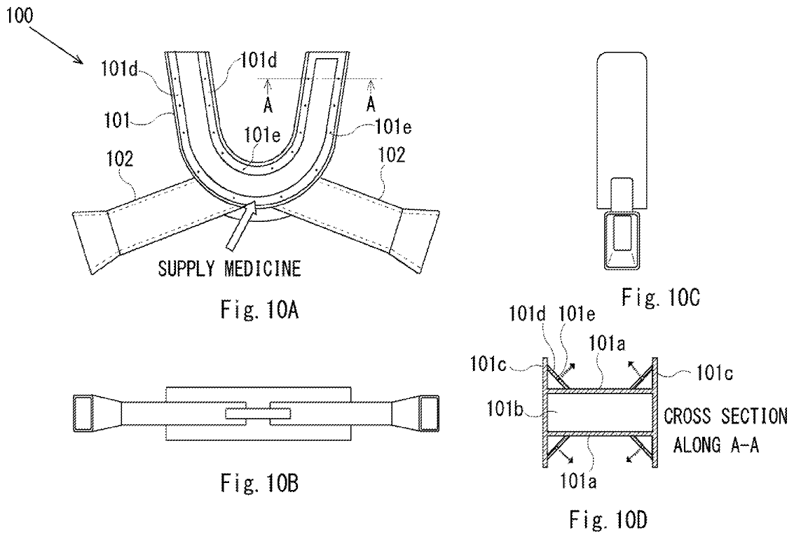

An example in which the guide member has a liquid supply function for supplying water, a medicinal solution, or the like to the oral tissues will now be explained with reference to FIG. 10A, FIG. 10B, FIG. 10C and FIG. 10D. FIG. 10A, FIG. 10B, FIG. 10C and FIG. 10D are schematic diagrams of a guide member 100 as the inspection auxiliary member. FIG. 10A is a plan view of the guide member 100, FIG. 10B is a front view, and FIG. 10C is a side view. FIG. 10D is a cross-sectional view taken along the line A-A in FIG. 10A. The guide member 100 includes a mouthpiece portion 101 to be held inside the mouth of the subject, and inside of which the imaging sensor moves, and an inserting and removing portion 102 via which the imaging sensor is inserted into or removed from the mouthpiece portion 101.

As illustrated in FIG. 10D, a tunnel-like path 101b having a rectangular cross section is formed inside the mouthpiece portion 101. The path 101b corresponds to a travel path, a path, or a guide. The top surface and the bottom surface of the path 101b in the orientation during the use include fixing portions 101a enabling the guide member 100 to be fixed, by being held inside of the mouth of the subject, between his/her maxillary and mandibular dentitions. In addition, provided on the ends of the path 101b in the horizontal direction are walls 101c by which the dentitions are covered from the buccal side (labial side) and the lingual side (pharynx side), respectively, when the subject holds the fixing portion 101a inside his/her mouth.

In the present embodiment, in the cross section A-A, tubular supply pipes 101d through which the water or a medicine can pass are provided on the left and the right ends of the upper and the lower fixing portions 101a of the path 101b (ends of the fixing portion 101a on the sides adjacent to the respective walls 101c). The supply pipe 101d provided on the outer circumferential side and the inner circumferential side of the path 101b may be connected to each other, on one end of the path 101b, as illustrated in FIG. 10A. The water or a medicinal solution then is injected into the supply pipe 101d from outside, at one position of the supply pipe 101d. The supply pipe 101d also includes a plurality of ejection holes 101e for ejecting the water or the medicinal solution, and the injected water or medicinal solution is ejected therefrom toward the oral tissues.

According to the present embodiment, it is possible to supply the water or a medicinal solution to the oral tissues, such as the dentition, using the guide member 100. Therefore, the oral tissues can be washed and treated efficiently. In the present embodiment, the medicinal solution ejected from the ejection holes 101e may also be liquid or gel.

### Sixth Embodiment

A sixth embodiment of the present application will be explained. In the present embodiment, imaging sensors are provided at a plurality of positions of the unit body. More specifically, imaging sensors are provided at appropriate positions of the unit body, at appropriate inclinations suitable for the position and direction of the oral tissues or the deposit that is an inspection target. With this, an appropriate imaging sensor can be selected and used depending on the position of the oral tissue, or the direction where an image is to be captured. With this, it is possible to broaden the range of the positions of the oral tissues and the directions from and in which an image can be captured. Furthermore, it becomes possible to capture images of a plurality of regions of oral tissues. Note that, in this configuration, the orientations of the imaging sensors may be inclined in any one of upwards, downwards, leftwards, and rightwards, or the imaging sensors may face horizontally or vertically.

Furthermore, in the present embodiment, a mirror that reflects the light or acoustic wave from the oral tissue or the deposit may be provided to the guide member, at an appropriate position of the path, the fixing portion, or the wall portion, and may guide the image of the oral tissue or the deposit to at least one of the imaging sensors that are provided at a plurality of positions of the unit body. With this, by selecting a combination of the imaging sensor and the mirror appropriately, it is possible to further broaden the range of the positions and direction at and in which images of the oral tissue or the deposit can be captured. The positions and angle of the mirror provided on the guide member may also be determined as appropriate, depending on the position and direction of the oral tissue or the deposit, and the position and direction of the imaging sensor to which the image is guided. The mirror may also be inclined in any one of upwards, downwards, leftwards, and rightwards, or face horizontally or vertically.

Explained in the embodiments above is an example in which an image of the oral tissue, particularly, the dentition is to be captured. However, the present invention may be applied to any device that emits light or ultrasonic waves to the oral tissue, and detects the reflected light, scattered light, fluorescence, luminescence, or the reflected waves of ultrasonic waves. In addition, the guide member as the inspection auxiliary member according to the present invention has the tunnel-like path, but the path does not need to have a tunnel-like shape. The guide member may have any shape, as long as the path can guide the movement of the imaging unit. In the present invention, the light from the oral tissue is reflected by the mirror and guided to the imaging sensor, but it is also possible to use an optical element that uses refraction, such as a prism, without limitation to a mirror.

Furthermore, the imaging unit or the guide member in the present invention may include a light source, and the detection light for inspection or the light for capturing image may be emitted to the oral tissue. Furthermore, it is also possible to disinfect the oral tissue by irradiating the oral tissues with ultraviolet rays or the like.

### REFERENCE SIGNS LIST

30, 50, 80, 100 guide member
31, 51, 81, 101 mouthpiece portion
32, 52, 82, 102 inserting and removing portion
35 image processing unit
36 determination unit
37 display unit
40, 60, 90 imaging unit
41, 61, 91 unit body
42, 62a to 62c, 92 imaging sensor
61a, 91a mirror
61d, 61f, 91d wheels
81d, 81e lateral mirror
91f driving wheel
94 drive actuator
101d supply pipe
101e ejection holes

1-21. (canceled)

22. An inspection device for oral tissues or deposits, the inspection device comprising:
a detection element for obtaining information on an oral tissue or a deposit that is an inspection target;
a case body internal of which is provided with the detection element or an information transmission line for transmitting information to the detection element; and
an inspection auxiliary member that includes a path or a guide along which the detection element or the information transmission line is movable, and that has a shape following a dentition.

23. The inspection device for oral tissues or deposits according to claim 22, wherein the inspection auxiliary member has a transmitting portion or a gap that transmits light or an acoustic wave from the oral tissue or the deposit.

24. The inspection device for oral tissues or deposits according to claim 22, further comprising a light source or an oscillator element disposed inside or outside the case body.

25. The inspection device for oral tissues or deposits according to claim 23, further comprising a light source or an oscillator element disposed inside or outside the case body, wherein
an oral cavity is irradiated with light from the light source or an acoustic wave from the oscillator element, via the path or the guide.

26. The inspection device for oral tissues or deposits according to claim 22, wherein the inspection auxiliary member or the case body is made from at least any one of a predetermined resin, ceramic, and metal that are safe for the oral tissues.

27. The inspection device for oral tissues or deposits according to claim 22, wherein the case body further includes a directing portion that reflects or refracts light or an acoustic wave from the oral tissue or the deposit that is the inspection target and directs the light or the acoustic wave to the detection element.

28. The inspection device for oral tissues or deposits according to claim 22, further comprising:
an image processing unit that generates an image or a spectrum of the oral tissue or the deposit that is the inspection target, based on an output signal from the detection element;
a determination unit that determines or aids determination of presence of a disease of an oral tissue that is the inspection target including a tooth, based on the image or the spectrum generated by the image processing unit; and
a display unit that displays the image or the spectrum generated by the image processing unit and/or a determination result of the determination unit.

29. The inspection device for oral tissues or deposits according to claim 22, wherein, the detection element is an imaging sensor that captures an image of the oral tissue that is the inspection target.

30. The inspection device for oral tissues or deposits according to claim 22, wherein
the case body is movable inside the inspection auxiliary member along the path or the guide in the inspection auxiliary member, and
a plurality of the detection elements are provided on a front side and lateral sides, respectively, with respect to a movement of the case body.

31. The inspection device for oral tissues or deposits according to claim 30, wherein
the case body includes a directing portion that is provided on a front side of the case body, being on the front side with respect to the movement, and that reflects or refracts light or an acoustic wave from the oral tissue or the deposit that is the inspection target and guides the light or the acoustic wave to the detection element, and
the inspection auxiliary member includes lateral directing portions that are provided to parts of the path or the guide in a manner facing the lateral sides of the case body, respectively, being on the lateral sides with respect to the movement, and that reflect or refract light or an acoustic wave from the oral tissue or the deposit that is the inspection target and guide the light or the acoustic wave to the detection element.

32. The inspection device for oral tissues or deposits according to claim 31, wherein
the detection element provided on the front side of the case body is installed facing a direction in which the case body is advanced, and the detection elements provided on the respective lateral sides of the case body are installed inclinedly with respect to an up-down direction of the case body, and
the directing portion, provided on the front side of the case body with respect to the movement, is installed inclinedly with respect to the up-down direction of the case body, and the lateral directing portions, provided to parts of the path or the guide of the inspection auxiliary member in a manner facing the lateral sides of the case body, respectively, being on the lateral sides with respect to the movement, are installed in an orientation in which normal lines of the lateral directing portions extend horizontally.

33. The inspection device for oral tissues or deposits according to claim 22, further comprising:
an image processing unit that generates an image of the oral tissue or the deposit that is the inspection target, based on an output signal from the detection element; and
a display unit that displays the image generated by the image processing unit, wherein
the oral tissue is a dentition,
the image processing unit generates a map image that is a combination of a linear image obtained by joining images of the dentition captured from a buccal side and/or a lingual side of the dentition to a linear image, and a planar image obtained by joining images of the dentition captured from an occlusal side to a planer image, the images of the dentition being captured using the detection element and the inspection auxiliary member, and
the display unit displays the map image.

34. The inspection device for oral tissues or deposits according to claim 22, wherein the path or the guide of the inspection auxiliary member further includes an ejector portion that ejects to supply a predetermined liquid or gel substance onto the oral tissue.

35. The inspection device for oral tissues or deposits according to claim 22, wherein the case body includes a propulsion mechanism for moving the case body along the path or the guide.

36. The inspection device for oral tissues or deposit according to claim 22,
wherein the case body moves along the path or the guide of the inspection auxiliary member, and
the inspection device further comprises a position information detection unit capable of obtaining position information of the case body inside the path or the guide.

37. The inspection device for oral tissues or deposits according to claim 22, wherein the detection element is provided at a plurality of positions on the case body.

38. The inspection device for oral tissues or deposits according to claim 37, wherein the inspection auxiliary member includes a directing portion that reflects or refracts light or an acoustic wave from the oral tissue or the deposit that is the inspection target and guides the light or the acoustic wave to at least one of the detection elements provided at the plurality of respective positions of the case body.

39. An inspection auxiliary member comprising a detection element that obtains information on an oral tissue or a deposit that is an inspection target, or a path or a guide through which an information transmission line is movable, and having a shape following a dentition.

40. The inspection auxiliary member according to claim 39, further comprising a transmitting portion or a gap that transmits light or an acoustic wave from the oral tissue or the deposit.

41. The inspection auxiliary member according to claim 39, wherein
the detection element moves along the path or the guide, and
the inspection auxiliary member includes a reflection surface that reflects light or an acoustic wave to be detected by the detection element, on a part of the shape following the dentition.
